(19) United States
(12) Patent Application Publication
(10) Pub. No.: US 2024/0407640 A1
(43) Pub. Date: Dec. 12, 2024

(54) Systems and Methods for Illumination Light Source Control

(71) Applicant: Alcon Inc., Fribourg (CH)

(72) Inventor: Ronald T. Smith, Irvine, CA (US)

(21) Appl. No.: 18/671,091

(22) Filed: May 22, 2024

Related U.S. Application Data
(60) Provisional application No. 63/507,520, filed on Jun. 12, 2023.
(60) Provisional application No. 63/594,898, filed on Oct. 31, 2023.

(51) Int. Cl. A61B 3/00; A61B 90/30

(52) U.S. Cl. CPC A61B 3/0008; A61B 3/0025; A61B 3/005; A61B 90/30

(57) ABSTRACT

Systems and methods for illumination light source control are provided. One method includes receiving a request to change an illumination output for one or more light sources, determining a total aphakic irradiance, determining a total aphakic irradiance normalized standard deviation, and determining a peak total aphakic irradiance based on the total aphakic irradiance and the total aphakic irradiance normalized standard deviation. The request includes a requested light level. When the peak total aphakic irradiance is less than or equal to an aphakic irradiance limit, the illumination output is changed based on the requested light level. When the peak total aphakic irradiance is greater than the aphakic irradiance limit, the requested light level is reduced until the peak total aphakic irradiance is less than or equal to the aphakic irradiance limit, and the illumination output is changed based on the reduced requested light level.

### BACKGROUND

The present disclosure relates to illumination light sources. More particularly, the present disclosure relates to systems and methods for illumination light source control.

Surgical procedures and diagnostic evaluations often use one or more illumination light sources to illuminate a particular region inside the body. For ocular surgery, it may be important to monitor and control the illumination provided to the interior of the eye to prevent damage to the retina, etc.

During many ocular procedures, the surgical team may use one or more endoilluminator light sources to directly illuminate the interior of the eye. The endoilluminator light source has a light guide that passes through a trocar cannula, access port, etc. that is attached to the sclera to directly illuminate the vitreous, the retina, etc. Because the illumination does not pass through the lens of the eye, the irradiance (radiant flux per unit area) provided by the endoilluminator light source is known as aphakic irradiance. Importantly, the aphakic irradiance provided by the endoilluminator light source should be limited to prevent damage to the retina. For example, the International Organization for Standardization (ISO) Standard 15752:2010 specifies an aphakic irradiance limit of 10 J/cm² (Joules per square centimeter) in 30 minutes or longer for a human retina.

### SUMMARY

Embodiments of the present disclosure advantageously provide systems and methods for illumination light source control.

In one embodiment, a method for controlling illumination light sources includes receiving a request to change an illumination output for one or more illumination light sources, determining a total aphakic irradiance (Eₐₛₜ) for the illumination light sources, determining a total aphakic irradiance normalized standard deviation (σ_{Eat}′) for the illumination light sources, and determining a peak total aphakic irradiance (E_{Ast}) based on the (average) total aphakic irradiance (Eₐₛₜ) and the total aphakic irradiance normalized standard deviation (σ_{Eat}′). The request to change an illumination output includes a requested light level (L_{requested}).

When the peak total aphakic irradiance (E_{Ast}) is less than or equal to an aphakic irradiance limit (E_{Limit}), the method includes changing the illumination output of the one or more illumination light sources based on the requested light level (L_{requested}).

When the peak total aphakic irradiance (E_{Ast}) at the requested light level L_{requested} would be greater than the aphakic irradiance limit (E_{Limit}), the method includes reducing the requested light level (L_{requested}) until the peak total aphakic irradiance (E_{Ast}) is less than or equal to the aphakic irradiance limit (E_{Limit}), and changing the illumination output of the one or more illumination light sources based on the reduced requested light level (L_{reduced}).

### BRIEF DESCRIPTION OF THE DRAWINGS

FIG. 1 illustrates an exemplary ophthalmic surgical system, in accordance with embodiments of the present disclosure.

FIG. 2A depicts a flow diagram illustrating functionality associated with controlling illumination light sources, in accordance with embodiments of the present disclosure.

FIGS. 2B, 2C, 2D, 2E depict flow diagrams illustrating functionality associated with controlling illumination light sources, in accordance with embodiments of the present disclosure.

FIG. 3 presents a graph illustrating an exemplary aphakic irradiance probability distribution, in accordance with embodiments of the present disclosure.

FIG. 4 depicts a graph of peak total aphakic irradiance over time for four illumination events, in accordance with embodiments of the present disclosure.

FIG. 5A depicts a graph of peak total aphakic exposure over time for four illumination events, in accordance with embodiments of the present disclosure.

FIGS. 5B, 5C, 5D depict graphs of peak total aphakic exposure over time, in accordance with embodiments of the present disclosure.

### DETAILED DESCRIPTION

Embodiments of the present disclosure will now be described with reference to the drawing figures, in which like reference numerals refer to like parts throughout.

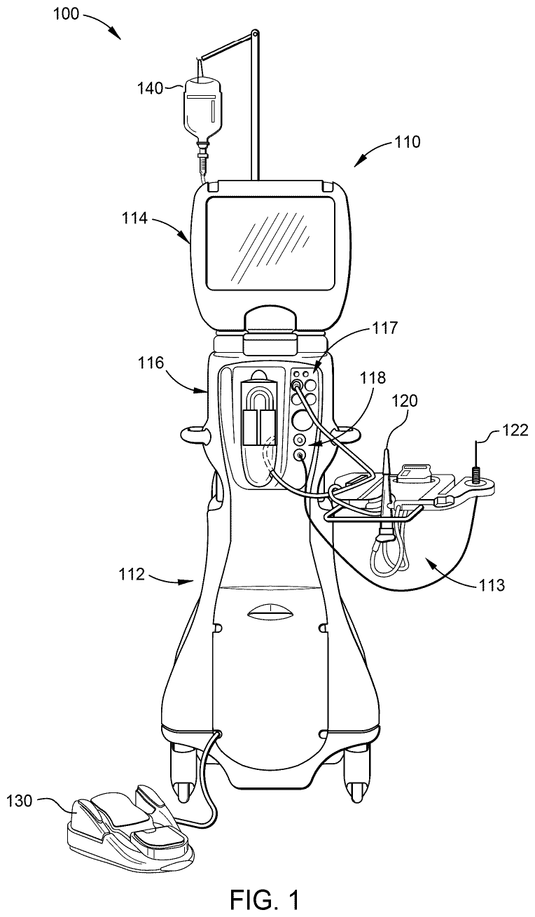

FIG. 1 illustrates ophthalmic surgical system 100, in accordance with embodiments of the present disclosure.

Ophthalmic surgical system 100 may include, inter alia, surgical console 110, one or more surgical tools 120, one or more illumination probes 122, one or more interface devices 130, one or more fluid reservoirs 140, etc.

Surgical console 110 may include, inter alia, a control system, housing 112, tray 113, display 114 (such as a touchscreen display, etc.), fluidics subsystem 116, surgical tool subsystem 117, illumination subsystem 118, etc.

The control system may include, inter alia, one or more processors, memory, communication interfaces, etc. The control system may be coupled to display 114, fluidics subsystem 116, surgical tool subsystem 117, illumination subsystem 118, and interface devices 130, and generally controls the operation of ophthalmic surgical system 100. More particularly, the control system may be configured to implement the methods for illumination light source control described herein.

In certain embodiments, the one or more processors may include a microprocessor, a controller (which may be a micro-controller), a digital signal processor (DSP), a microcomputer, a central processing unit (CPU), field programmable gate array (FPGA), programmable logic device (PLD), state machine, logic circuitry, control circuitry, analog circuitry, digital circuitry, etc., as well as any combination of these devices. Generally, the processor may be any device that manipulates signals (analog and/or digital) based on operational instructions.

The memory may be coupled to, and/or embedded within, the one or more processors. The memory may be a single memory device, multiple memory devices, or a combination of memory devices, such as read-only memory (ROM), random access memory (RAM), volatile memory, non-volatile memory, static memory, dynamic memory, flash memory, cache memory, etc. Generally, the memory may be any device that stores digital information, such as operational instructions and data corresponding to at least some of the elements illustrated and described in association with the figures.

Fluidics subsystem 116 may be coupled to one or more surgical tools 120. Fluidics subsystem 116 includes, inter alia, fluid reservoirs 140 and various other components, such as fluid lines (tubes), connections, control valves, etc. Fluid reservoirs 140 may include irrigation fluid reservoirs, aspiration fluid reservoirs, etc., and may be located outside of housing 112 (as depicted) or inside of housing 112.

Surgical tool subsystem 117 may be coupled to surgical tools 120 to provide the appropriate interfaces for each surgical tool 120, such as electrical power, control and communication signals, mechanical connections, hydraulic connections, pneumatic connections, ultrasonic signals, etc.

Illumination subsystem 118 includes one or more illumination light sources (not depicted for clarity), such as an endoilluminator light source, a light emitting diode (LED), red, green and blue LEDs optically multiplexed into a white beam, a white LED, etc. Generally, an illumination light source is coupled to an illumination probe 122. In certain embodiments, illumination subsystem 118 may include, inter alia, a controller (such as a processor, microprocessor, etc.) that may be configured to implement at least a portion of the methods for illumination light source control described herein.

Surgical tools 120 may include, inter alia, a vitrectomy probe, a phacoemulsification probe, etc.

Illumination probes 122 may have a needle, a handle, and a fiber optic cable that couples the needle to an illumination light source within illumination subsystem 118. Illumination probes 122 may include, inter alia, an endoilluminator light probe or guide, an illuminated pik (or pick), an illuminated laser probe, etc. In certain embodiments, a surgical tool 120 may have an integrated illumination probe 122 that is coupled to an illumination light source.

Interface devices 130 may include a foot pedal with one or more pedals (as depicted), a joystick, a touch pad, a mouse, a keyboard, etc.

Surgical console 110 is merely an exemplary depiction of a console for illustration purposes, and the embodiments described herein are applicable to a variety of consoles that may look or function similarly or differently in relation to surgical console 110. Further, the embodiments described herein may be equally applicable to diagnostic equipment providing illumination to, for example, the eye. Other surgical consoles and diagnostic equipment are also contemplated, such as for use with other body parts that may be sensitive to the amount of illumination being provided.

The control system may control one or more illumination light sources in accordance with certain constraints. For example, during vitreoretinal surgery, ISO Standard 15752:2010 (read in combination with ISO Standard 15004-2:2007) requires that a patient's retina not be exposed to an aphakic irradiance level of more than 10 J/cm² (Joules per square centimeter) in 30 minutes (or longer). Additionally, if the normal operating mode exceeds the aphakic irradiance limit, ISO Standard 15752:2010 requires that the illumination sub-system provide a user-optional retinal protection operating mode that does meet the aphakic irradiance limit.

In certain embodiments, control system may control the illumination light sources in accordance with certain ISO standards. For constant illumination, the ISO standard aphakic irradiance exposure level of 10 J/cm² in 30 minutes (or longer) is equivalent to an aphakic irradiance limit (EA-ISO) of 5.556 mW/cm² (milliwatts per square centimeter).

While the aphakic irradiance limit (EA-ISO) is provided as an example, it is to be understood that the disclosed systems and methods may be used with other aphakic irradiance limits, such as a different aphakic irradiance limit set by a different standard, an aphakic irradiance limit provided by user input (via touchscreen display 114, etc.), etc. Further, while the examples provided herein control one or more illumination light sources in accordance with aphakic irradiance limits, the same techniques can be applied to monitor and control illumination light sources to prevent exceeding, for example, a retina thermal irradiance limit or other standard or user provided limits.

In certain embodiments, illumination parameters may be measured, calculated, determined, etc., and then stored for the illumination light sources used in the surgical procedure or diagnostic evaluation. The illumination parameters for each illumination light source may include, for example, average (over the distribution of manufactured illumination systems) aphakic irradiance (Eₐₛ) in saline solution (as specified by ISO Standard 15752:2010), aphakic irradiance standard deviation σ_{Ea}, and aphakic irradiance normalized standard deviation (σ_{Ea}′).

The aphakic irradiance Eₐₛ for each illumination probe at the light level (L) set by the user is given by:

$${E}_{as} = ( L / {L}_{max} ) \cdot {E}_{as - max} \qquad Eq . 1$$

where Eₐₛ₋ₘₐₓ (also known as I_{A-ave-max}) refers to the (average) aphakic irradiance (Eₐₛ) at the maximum light level (Lₘₐₓ) for each illumination probe, and L/Lₘₐₓ is known as the light level ratio. The aphakic irradiance normalized standard deviation (σ_{Ea}′) is given by:

$${σ}_{Ea}^{′} = {σ}_{Ea} / {E}_{as} \qquad Eq . 2$$

In certain embodiments, the illumination parameters may be determined for a particular illumination output or light level (L) during the surgical procedure. In certain embodiments, the instructions for use (IFU) for an illumination probe may specify a particular working distance (WD), and the illumination parameters may be determined relative to the WD.

For example, four (4) illumination probes 122 may be coupled to four (4) illumination light sources, and the user may individually (or collectively) set the illumination output or light level (L) of each illumination light source. The illumination parameters may include:
L₁, L₂, L₃, L₄, Lₘₐₓ₁, Lₘₐₓ₂, Lₘₐₓ₃, Lₘₐₓ₄;
   Eₐₛ₋ₘₐₓ₁, Eₐₛ₋ₘₐₓ₂, Eₐₛ₋ₘₐₓ₃, Eₐₛ₋ₘₐₓ₄, Eₐₛ₁, Eₐₛ₂, Eₐₛ₃, Eₐₛ₄;
      σ_{Ea1}, σ_{Ea2}, σ_{Ea3}, σ_{Ea4}, σ_{Ea1}′, σ_{Ea2}′, σ_{Ea3}′, σ_{Ea4}′.

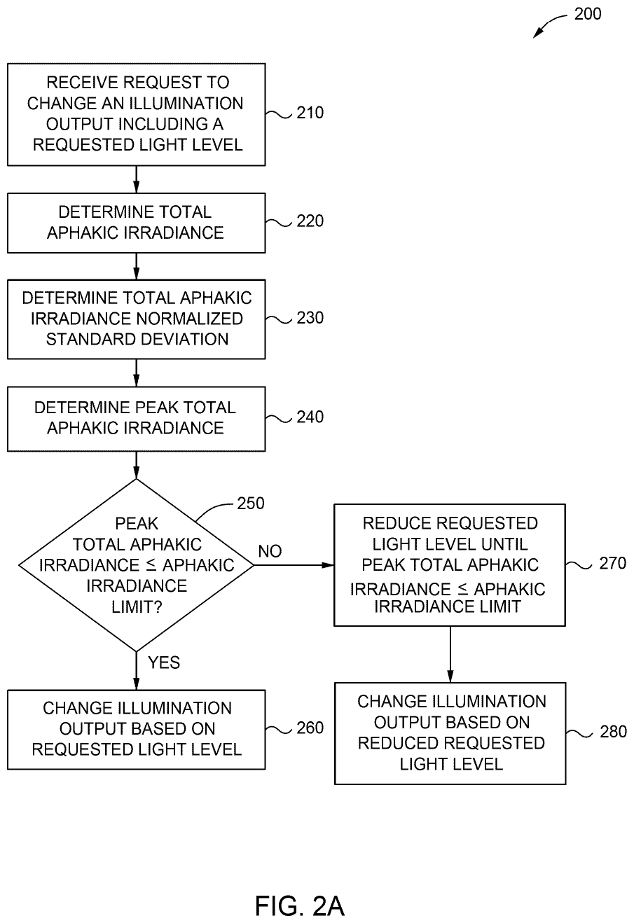

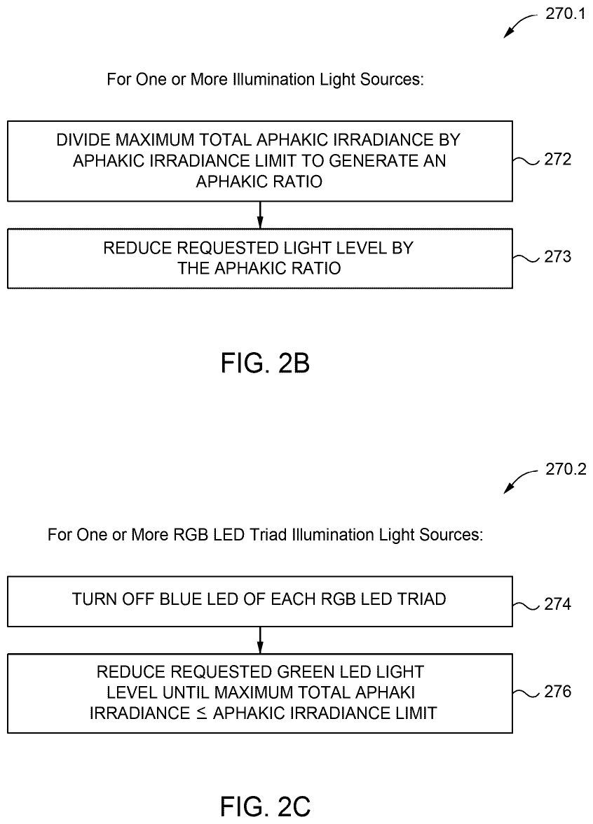

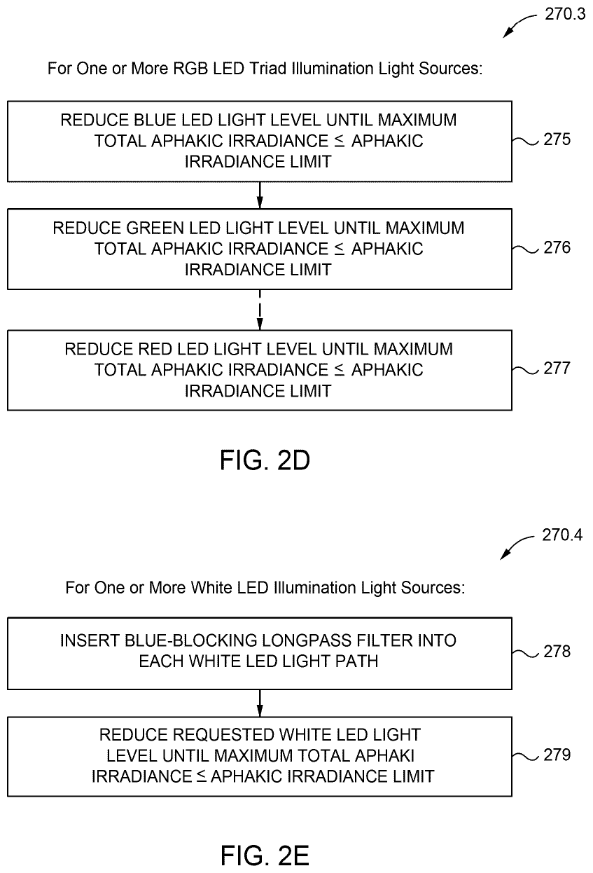

FIG. 2A depicts flow diagram 200 illustrating functionality associated with controlling illumination light sources, in accordance with embodiments of the present disclosure.

Generally, the functionality associated with controlling the illumination light sources is performed by the control system and illumination subsystem 118 when one or more illumination light sources are turned on during the surgical procedure. In certain embodiments, the functionality associated with controlling illumination light sources may be enabled and disabled by the user through a graphical user interface (GUI) displayed on a touchscreen display 114, activation of a control of an interface device 130 (such as a foot pedal, etc.), etc.

At 210, a request to change the illumination output for one or more illumination light sources is received. The request includes a requested light level (L_{Requested}).

In certain embodiments, the control system may receive a request from the user to change the illumination output or light level (L_{Requested}) of one or more illumination light sources of illumination subsystem 118, such as a request to increase the illumination output (up to Lₘₐₓ) or a request to decrease the illumination output (down to 0).

For example, the user may request a new illumination output or light level (L_{Requested}) for a particular illumination probe 122 by selecting, setting, manipulating, etc., a respective GUI widget displayed on a touchscreen display 114, activating a respective control of an interface device 130 (such as a foot pedal, etc.), etc. In another example, the user may request a new illumination output or light level (L_{Requested}) for all of the illumination probes 122 by selecting, setting, manipulating, etc., a single (collective) GUI widget displayed on a touchscreen display 114, activating a single (collective) control of an interface device 130 (such as a foot pedal, etc.), etc. In a further example, the handpiece of each illumination probe 122 may include a control (such as a button, a slider, etc.) that sends a control signal to the control system to request a new illumination output or light level (L_{Requested}). In all these examples, the request is received by the control system through the communication interface.

At 220, the total aphakic irradiance (Eₐₛₜ) corresponding to L_{Requested} for the one or more illumination light sources is determined.

In certain embodiments, the control system calculates the aphakic irradiance at the maximum light level (Lₘₐₓ) for each illumination probe (also known as the maximum light level aphakic irradiance or Eₐₛ₋ₘₐₓ), and then calculates the aphakic irradiance (Eₐₛ) for each illumination probe (as given by Equation 1). The control system then calculates the total aphakic irradiance (Eₐₛₜ) by summing the aphakic irradiance (Eₐₛ) for each illumination probe 122.

As discussed above, the requested light level (L_{Requested}) may apply to each illumination probe 122 (i.e., a collective request), and the aphakic irradiance (Eₐₛ) for each illumination probe is calculated for the requested light level (L_{Requested}). Alternatively, the requested light level (L_{Requested}) may apply to one of the illumination probes 122, while the other illumination probes 122 remain set to their current light levels (L).

For the four illumination probe example described above, the total aphakic irradiance Eₐₛₜ is given by:

$${E}_{ast} = {E}_{as 1} + {E}_{as 2} + {E}_{as 3} + {E}_{as 4} \qquad Eq . 3$$

At 230, the total aphakic irradiance normalized standard deviation (σ_{Eat}′) for the one or more illumination light sources is determined.

In certain embodiments, the control system calculates, or retrieves from a stored database, the aphakic irradiance standard deviation (σ_{Ea}) for each illumination probe, and then calculates aphakic irradiance normalized standard deviation (σ_{Ea}′) for each illumination probe (as given by Equation 2). The control system then calculates the total aphakic irradiance normalized standard deviation (σ_{Eat}′) by calculating the square root of the sum of the squares of the aphakic irradiance normalized standard deviation (σ_{Ea}′) for each illumination light source.

For the four illumination probe example described above, the total aphakic irradiance normalized standard deviation (σ_{Eat}′) for four simultaneously illuminated probes may be calculated as:

$${σ}_{Eat}^{′} = \sqrt{{σ}_{Ea 1}^{′2} + {σ}_{Ea 2}^{′2} + {σ}_{Ea 3}^{′2} + {σ}_{Ea 4}^{′2}} \qquad Eq . 4$$

At 240, the peak total aphakic irradiance (E_{Ast}) is determined based on the total aphakic irradiance (Eₐₛₜ) and the total aphakic irradiance normalized standard deviation (σ_{Eat}′).

In certain embodiments, the control system calculates the highest or peak total aphakic irradiance E_{Ast} as follows:

$${E}_{Ast} = {E}_{ast} \cdot ( 1 + n \cdot {σ}_{Eat}^{′} ) \qquad Eq . 5$$

where “n” is a safety factor (i.e., the number of normalized standard deviations) that is selected to achieve an upper limit on the fraction of cases where the “actual” total aphakic irradiance is expected to exceed the “peak” total aphakic irradiance (E_{Ast}). For example, n may be selected as 4.75, which results in 1 in 1,000,000 cases where the actual total aphakic irradiance may exceed the peak total aphakic irradiance (E_{Ast}). Other values for n may also be selected. In some certain embodiments, n may be entered by the user using a GUI displayed on a touchscreen display 114.

At 250, the control system compares the peak total aphakic irradiance (E_{Ast}) to the aphakic irradiance limit (E_{Limit}), such as an aphakic irradiance limit (EA-ISO) of 5.556 mW/cm².

When the peak total aphakic irradiance (E_{Ast}) is less than or equal to the aphakic irradiance limit (E_{Limit}), the flow proceeds to 260, and when the peak total aphakic irradiance (E_{Ast}) is greater than the aphakic irradiance limit (E_{Limit}), the flow proceeds to 270.

At 260, the peak total aphakic irradiance (E_{Ast}) corresponding to the implementation of L_{Requested} is less than or equal to the aphakic irradiance limit (E_{Limit}), and the control system changes the illumination output of the one or more illumination light sources to the requested light level (L_{Requested}).

In other words, the control system has determined that the requested light level (L_{Requested}) should not cause the peak total aphakic irradiance (E_{Ast}) to exceed the aphakic irradiance limit (E_{Limit}), so the request to change the illumination output for one or more illumination light sources may be processed.

For example, when the request is associated with a single illumination probe 122 and illumination light source, the control system may send a command to illumination subsystem 118 to change the illumination output of the illumination light source to the requested light level (L_{Requested}). For another example, when the request is associated with all of illumination probes 122 and illumination light sources, the control system may send a command to illumination subsystem 118 to change the illumination output of all of the illumination light sources to the requested light level (L_{Requested}).

At 270, the peak total aphakic irradiance (E_{Ast}) is greater than the aphakic irradiance limit (E_{Limit}). The control system reduces the requested light level (L_{Requested}) until the peak total aphakic irradiance (E_{Ast}) is less than or equal to the aphakic irradiance limit (E_{Limit}).

In other words, the control system has determined that the requested light level (L_{Requested}) may cause the peak total aphakic irradiance (E_{Ast}) to exceed the aphakic irradiance limit (E_{Limit}), so the requested light level (L_{Requested}) is reduced until the peak total aphakic irradiance (E_{Ast}) is less than or equal to the aphakic irradiance limit (E_{Limit}). The reduced requested light level (L_{Reduced}) is then processed at 280.

In certain embodiments, the user may be notified that a reduction is being applied to the requested light level (L_{Requested}) to lower the illumination output (i.e., the peak total aphakic irradiance (E_{Ast})) so that the aphakic irradiance limit (E_{Limit}) is not exceeded, such as a notification presented on a GUI displayed on the touchscreen display 114.

In certain embodiments, the control system may be configured to provide an option to the user, presented via the GUI, to override the reduction of the requested light level (L_{Requested}) for a particular illumination probe 122. If the user selects the override option, the control system may be configured to reduce the light levels (L) of the other illumination light sources until the peak total aphakic irradiance (E_{Ast}) is less than or equal to the aphakic irradiance limit (E_{Limit}) for all of the illumination light sources (i.e., including the requested light level (L_{Requested}) for the particular illumination probe 122).

At 280, the control system changes the illumination output of the one or more illumination light sources based on the reduced requested light level (L_{Reduced}).

Similar to the process discussed with respect to 260, when the request is associated with a single illumination probe 122 and illumination light source, the control system may send a command to illumination subsystem 118 to change the illumination output of the illumination light source to the reduced requested light level (L_{Reduced}). And, when the request is associated with all of the illumination probes 122 and illumination light sources, the control system may send a command to illumination subsystem 118 to change the illumination output of all of the illumination light sources to the reduced requested light level (L_{Reduced}).

In certain embodiments, the control system generally assumes that the peak total aphakic irradiance (E_{Ast}) is less than or equal to the aphakic irradiance limit (E_{Limit}, such as EA-ISO) before a user request is received. When a user request is received, the control system determines whether or not the relationship E_{Ast}≤ E_{Limit} would be maintained at the requested light level (L_{Requested}). If the relationship would be maintained, then the user request is granted, and the control system changes the illumination output of the one or more illumination light sources to the requested light level (L_{Requested}), as described above. If the relationship would not be maintained (i.e., E_{Ast}>E_{Limit}), then the control system takes steps to ensure that the relationship E_{Ast}≤ ELinit is achieved, as described above. In other embodiments, the control system may periodically determine whether the peak total aphakic irradiance (E_{Ast}) is less than or equal to the aphakic irradiance limit (E_{Limit}, such as EA-ISO), and, if the peak total aphakic irradiance (E_{Ast}) is greater than the aphakic irradiance limit (E_{Limit}), then the control system may take steps to ensure that the relationship E_{Ast}≤ E_{Limit} is achieved, as described above.

FIG. 2B depicts flow diagram 270.1 illustrating functionality associated with controlling illumination light sources, in accordance with embodiments of the present disclosure.

When the peak total aphakic irradiance (E_{Ast}) corresponding to a requested light level (L_{Requested}) would be greater than the aphakic irradiance limit (E_{Limit}), the control system reduces the requested light level (L_{Requested}) until the peak total aphakic irradiance (E_{Ast}) is less than or equal to the aphakic irradiance limit (E_{Limit}), as described above with respect to 270.

Flow diagram 270.1 depicts an example of the functionality described at 270.

At 272, the peak total aphakic irradiance (E_{Ast}) is divided by the aphakic irradiance limit (E_{Limit}) to generate the aphakic irradiance ratio (E_{Ast}/E_{Limit}).

At 273, the requested light level (L_{Requested}) is divided by the aphakic irradiance ratio (E_{Ast}/E_{Limit}) to generate the reduced requested light level (L_{Reduced}).

In other words, the control system may reduce the illumination output (or in some other way reduce an illumination level) of the one or more illumination light sources by the aphakic irradiance ratio (E_{Ast}/E_{Limit}). For example, when the radiometric flux output of each illumination light source is linear with respect to the input electrical power, then the electrical power supplied to the one or more illumination light sources may be divided by the aphakic irradiance ratio (E_{Ast}/E_{Limit}) to reduce the illumination output.

FIG. 2C depicts flow diagram 270.2 illustrating functionality associated with controlling illumination light sources, in accordance with embodiments of the present disclosure.

Flow diagram 270.2 depicts another example of the functionality described at 270 for illumination light sources that include RGB (Red Green Blue) LED triads.

Generally, light having a wavelength in the blue portion of the spectrum (about 380 nm (nanometers) to 500 nm) presents a higher aphakic hazard that light in the green portion of the spectrum (about 495 nm to 570 nm). Light in the red portion of the spectrum (about 620 nm to 750 nm) presents little aphakic hazard. Accordingly, the chromaticity of the light output by the illumination light sources may be adjusted based on the relative aphakic hazard of each major color wavelength band.

At 274, the blue LED light level (L_{B}) of each illumination light source is reduced to zero.

At 276, the green LED light level (L_{G}) of each illumination light source is reduced until the peak total aphakic irradiance (E_{Ast}) is less than or equal to the aphakic irradiance limit (E_{Limit}) to generate the reduced requested light level (L_{Reduced}).

In other words, the control system may eliminate the blue component of the illumination output of the illumination light source, and then reduce the green component of the illumination output of the illumination light source until the peak total aphakic irradiance (E_{Ast}) is less than or equal to the aphakic irradiance limit (E_{Limit}). The red component of the illumination output of the illumination light source may remain at its original setting and may provide a significant portion of the illumination of the retina.

Since the photopic eye response function V(λ), where λ is wavelength, peaks at 555 nm and rolls off rapidly on either side of the peak wavelength, green light, which is around 515-560 nm, has a high luminous flux to radiometric flux ratio, but blue light, which is below 500 nm, has a low luminous flux to radiometric flux ratio. Furthermore, since the aphakic sensitivity function A(λ) increases rapidly towards lower visible wavelengths and into the ultraviolet, blue light has a high aphakic flux to radiometric flux ratio, while green light has a much lower aphakic flux to radiometric flux ratio. Therefore, blue light has a much higher aphakic flux to luminous flux ratio than green light. Therefore, eliminating blue light first is a good strategy for reducing aphakic irradiance (aphakic flux per unit area) dramatically while reducing the illuminance (luminous flux per unit area) only slightly, thereby providing plenty of visible illumination during surgery.

FIG. 2D depicts flow diagram 270.3 illustrating functionality associated with controlling illumination light sources, in accordance with embodiments of the present disclosure.

Flow diagram 270.3 depicts another example of the functionality described at 270 for illumination light sources that include RGB LED triads.

At 275, the blue LED light level (L_{B}) of each illumination light source is reduced until the peak total aphakic irradiance (E_{Ast}) is less than or equal to the aphakic irradiance limit (E_{Limit}) to generate the reduced requested light level (L_{Reduced}). If the blue LED light level (L_{B}) is reduced to 0 and the peak total aphakic irradiance (E_{Ast}) is still greater than the aphakic irradiance limit (E_{Limit}), then flow proceeds to 276.

At 276, the green LED light level (L_{G}) of each illumination light source is reduced until the peak total aphakic irradiance (E_{Ast}) is less than or equal to the aphakic irradiance limit (E_{Limit}) to generate the reduced requested light level (L_{Reduced}), as described above.

In other words, the control system may reduce the blue component of the illumination source, and then reduce the green component of the illumination source. More particularly, the control system may reduce the blue component until the peak total aphakic irradiance (E_{Ast}) is less than or equal to the aphakic irradiance limit (E_{Limit}). If the blue component is reduced to 0 and the peak total aphakic irradiance (E_{Ast}) is still greater than the aphakic irradiance limit (E_{Limit}), then the control system may reduce the green component until the peak total aphakic irradiance (E_{Ast}) is less than or equal to the aphakic irradiance limit (E_{Limit}) (similar to flow diagram 270.2).

In certain embodiments, the control system may also adjust the red LED light level (L_{R}) of each illumination light source (even though the red component presents little aphakic hazard). Accordingly, if the green LED light level (L_{G}) is reduced to 0 and the peak total aphakic irradiance (E_{Ast}) is still greater than the aphakic irradiance limit (E_{Limit}), then flow may proceed from 276 to 277.

At 277, the red LED light level (L_{R}) of each illumination light source is reduced until the peak total aphakic irradiance (E_{Ast}) is less than or equal to the aphakic irradiance limit (E_{Limit}) to generate the reduced requested light level (L_{Reduced}).

In other words, if the green component is reduced to 0 and the peak total aphakic irradiance (E_{Ast}) is still greater than the aphakic irradiance limit (E_{Limit}), then the control system may reduce the red component until the peak total aphakic irradiance (E_{Ast}) is less than or equal to the aphakic irradiance limit (E_{Limit}). In another example, the control system may reduce both the green and red components of the illumination output of the illumination light source at the same time until the peak total aphakic irradiance (E_{Ast}) is less than or equal to the aphakic irradiance limit (E_{Limit}). In a further example, the control system may reduce both the green and red components of the illumination output of the illumination light source by a user-defined ratio to ensure that the peak total aphakic irradiance (E_{Ast}) is less than or equal to the aphakic irradiance limit (E_{Limit}).

FIG. 2E depicts flow diagram 270.4 illustrating functionality associated with controlling illumination light sources, in accordance with embodiments of the present disclosure.

Flow diagram 270.4 depicts another example of the functionality described at 270 for illumination light sources that include a white LED.

At 278, a blue-blocking longpass filter is inserted into the light path of each illumination light source.

At 279, the white LED light level (L_{W}) of each illumination light source is reduced until the peak total aphakic irradiance (E_{Ast}) is less than or equal to the aphakic irradiance limit (E_{Limit}) to generate the reduced requested light level (L_{Reduced}).

In other words, the control system may reduce (or eliminate) the blue component of the illumination output of the white LEDs, and then reduce the illumination output until the peak total aphakic irradiance (E_{Ast}) is less than or equal to the aphakic irradiance limit (E_{Limit}). In certain embodiments, other filter types may be used in addition to or in place of the blue-blocking longpass filter, such as an infrared filter, an ultraviolet filter, etc.

The elements provided in the flow diagrams are illustrative only. Various elements depicted therein and described herein may be omitted, additional elements may be added, and various elements may be performed in a different order than provided.

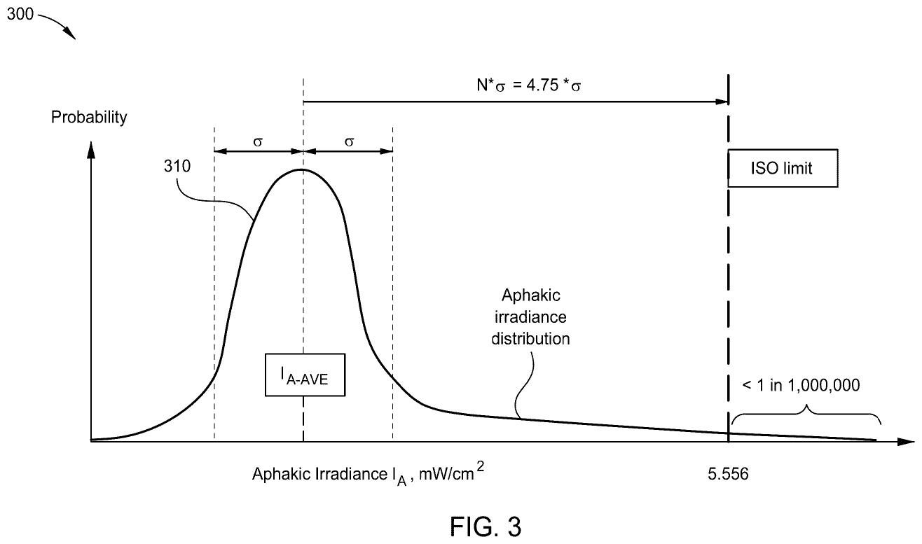

FIG. 3 presents graph 300 illustrating aphakic irradiance probability distribution 310, in accordance with embodiments of the present disclosure. Aphakic irradiance (I_{A}) is presented on the X-axis, while probability is presented on the Y-axis.

As described above, the control system generally analyzes and determines the proper response to a request to change the illumination output for the one or more illumination light sources from a user. For constant illumination, the ISO standard aphakic irradiance exposure level of 10 J/cm² in 30 minutes (or longer) is equivalent to an aphakic irradiance limit (EA-ISO) of 5.556 mW/cm² (milliwatts per square centimeter), so the acceptance condition may be expressed as:

$${I}_{A - ave} + 4.75 \cdot σ \leq 5.556 W / {cm}^{2} \qquad Eq . 6$$

In this example, “n” is 4.75, which results in 1 in 1,000,000 cases where the actual total aphakic irradiance may exceed the 5.556 W/cm².

In certain embodiments, the color rendering index (CRI) and color temperature (CCT) of the output of the illumination probe 122 may depend on user-selected chromaticity; other standard or user-defined system requirements may also be influenced by the control of chromaticity. In certain embodiments, the control system may be configured to screen user chromaticity requests and only permit chromaticities that meet the CRI and/or CCT or other requirements. Additionally, in the event the requested chromaticity was not acceptable, the control system may automatically select the closest point in chromaticity space to the requested chromaticity that is acceptable with respect to the aphakic irradiance limit (E_{Limit}).

In certain embodiments, the control system may be configured to determine whether a requested light level (L_{Requested}) for a particular illumination probe 122 will increase the luminous flux to a level that is higher than the thermal tip melting limit for that particular illumination probe 122. For example, a heavily contaminated plastic-fiber probe above the thermal tip melting limit could potentially degrade or melt, thereby damaging the patient's eye. If the requested light level would exceed the thermal tip melting limit, the software provides an advisory to allow the user to continue with the requested light level (L_{Requested}), to reduce the requested light level (L_{Requested}), etc.

Advantageously, in addition to controlling the illumination light sources of ophthalmic surgical system 100 (as described above), the control system may monitor the exposure of the patient to certain medical hazards and provide notifications to the surgeon as the surgical procedure progresses. In certain embodiments, the control system may be configured to determine and display aphakic and thermal hazard notifications, such as those required by one or more relevant standard(s), which may include aphakic hazard metrics, thermal hazard metrics, etc. For example, the control system may be configured to determine and present one or more hazard metrics to the user via a GUI displayed on touchscreen display 114, such as an aphakic figure of merit (M), an aphakic hazard metric, a thermal hazard metric, etc. In certain embodiments, the thermal hazard metric may include a remaining thermal exposure time (tₜₕₑᵣₘₐₗ) before the recommended maximum thermal exposure to the retina is exceeded. Other medical hazard metrics are also supported.

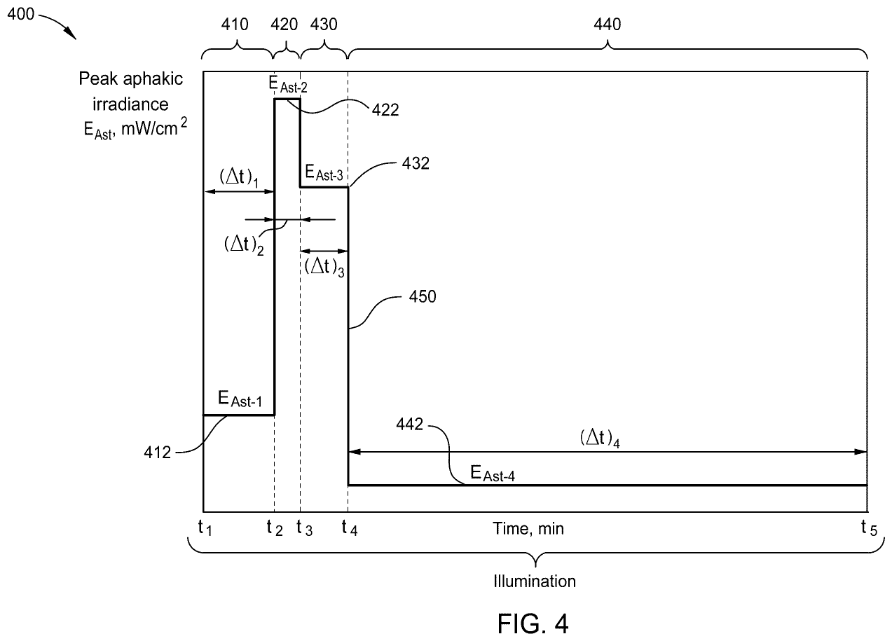

FIG. 4 depicts a graph 400 of peak total aphakic irradiance (E_{Ast}) 450 over time for four illumination events. Time (minutes) is presented on the X-axis, while peak total aphakic irradiance (E_{Ast}, mW/cm²) is presented on the Y-axis.

The first illumination event 410 begins at t₁ and ends at t₂ (Δt₁) and has a constant peak total aphakic irradiance (E_{Ast-1}) 412. The first illumination event 410 may represent the operation of one (1) illumination probe 122 and illumination light source at a particular light level.

The second illumination event 420 begins at t₂ and ends at t₃ (Δt₂) and has a constant peak total aphakic irradiance (E_{Ast-2}) 422. The second illumination event 420 may represent the operation of two (2) illumination probes 122 and illumination light sources at higher light levels.

The third illumination event 430 begins at t₃ and ends at t₄ (Δt₃) and has a constant peak total aphakic irradiance (E_{Ast-3}) 432. The third illumination event 430 may represent the operation of three (3) illumination probes 122 and illumination light sources at intermediate light levels.

The fourth illumination event 440 begins at t₄ and ends at t₅ (Δt₄) and has a constant peak total aphakic irradiance (E_{Ast-4}) 442. The fourth illumination event 440 may represent the operation of one (1) illumination probe 122 and illumination light source at a lower light level.

Generally, the control system may integrate the peak total aphakic irradiance (E_{Ast}) over time to generate a peak total aphakic exposure (H_{Ast}). In other words, peak total aphakic exposure (H_{Ast}) is a cumulative value that may be used to determine the aphakic exposure risk for the patient.

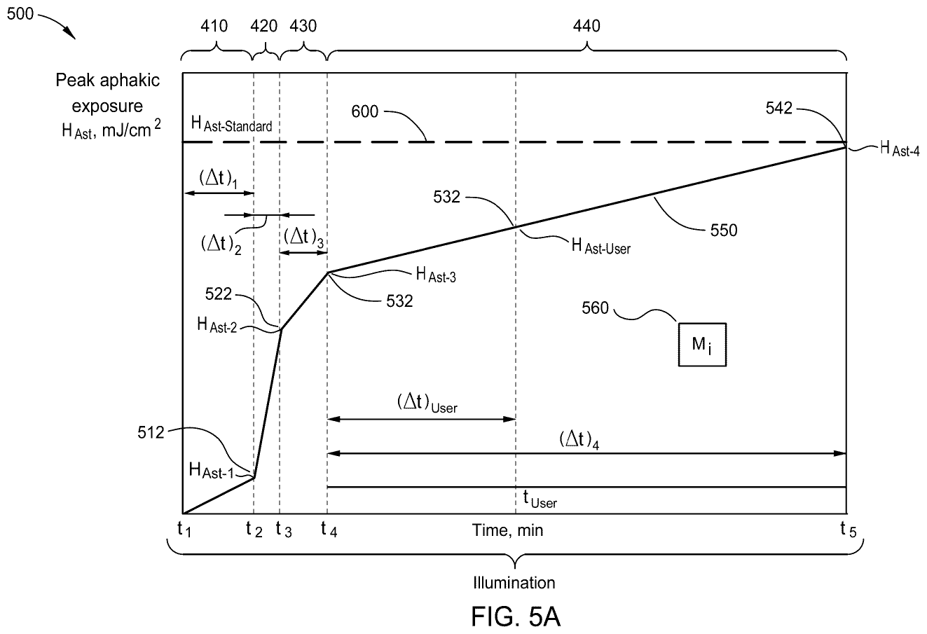

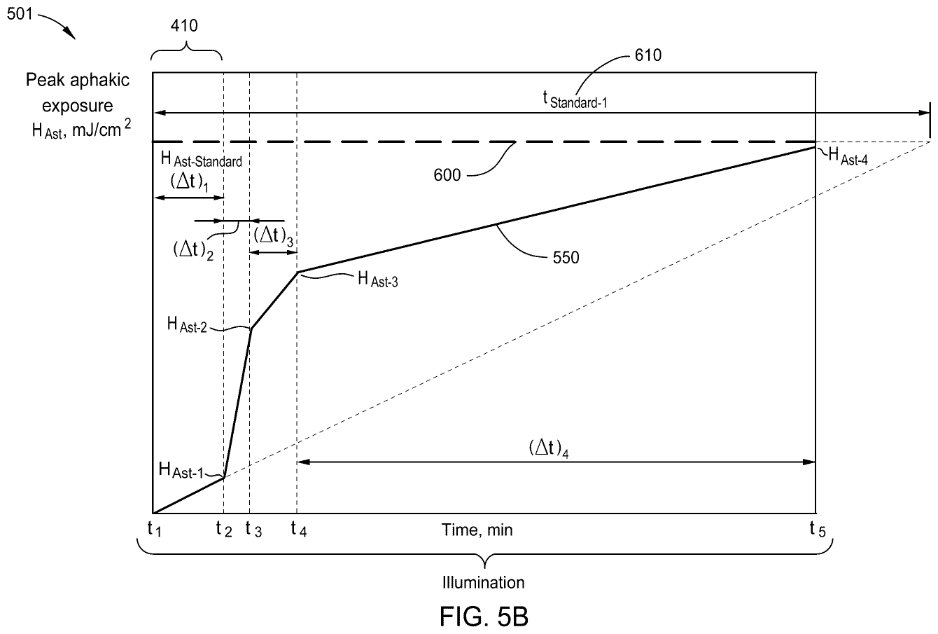

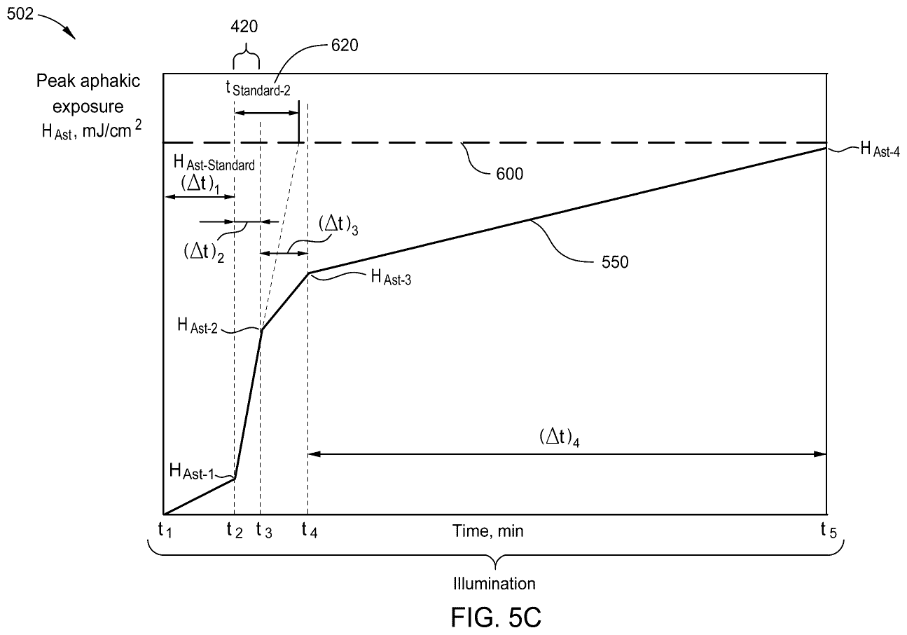

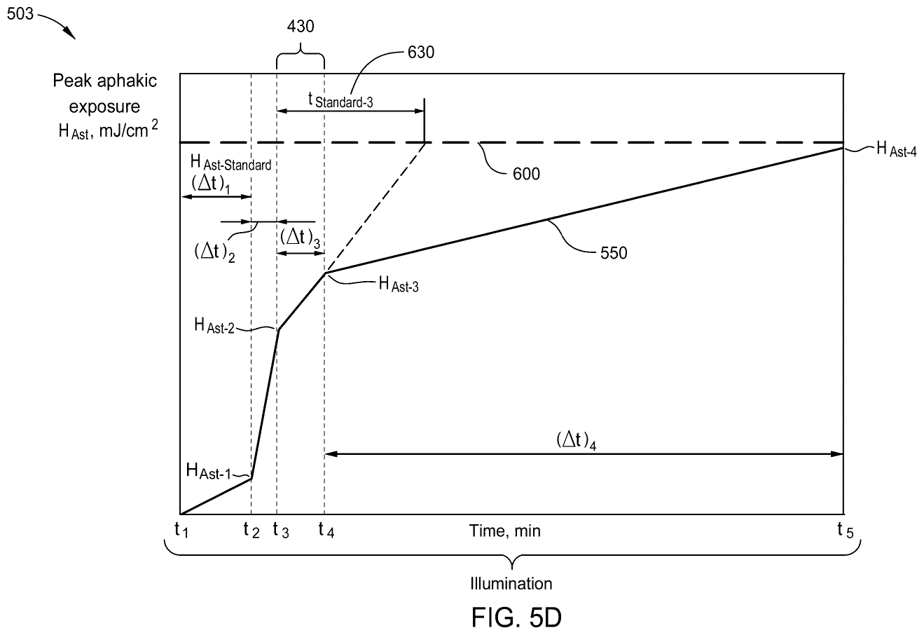

FIG. 5A depicts a graph 500 of the peak total aphakic exposure (H_{Ast}) 550 over time for four illumination events. Time (minutes) is presented on the X-axis, while peak total aphakic exposure (H_{Ast}, mJ/cm²) (milli-Joules per square centimeter) is presented on the Y-axis.

In certain embodiments, the peak total aphakic exposure (H_{Ast}) may be calculated from the time that a first illumination probe is turned on (t₁) until certain illumination events (or transitions) occur during the procedure, such as increasing the light level of the first illumination probe and turning on a second illumination probe (such as t₂), turning on a third illumination probe while reducing the light level of the first and second probes (such as t₃), turning off the second and third illumination probes while decreasing the light level of the first illumination probe (such as t₄), turning off the first illumination probe (such as t₅), etc. Additionally, the peak total aphakic exposure (H_{Ast}) may also be calculated from the time that a first illumination probe is turned on (t₁) until a request is received from the user (such as t_{User}). Further, an aphakic figure of merit (M) may be determined for each calculated peak total aphakic exposure (H_{Ast}) by dividing the peak total aphakic exposure (H_{Ast}) by the relevant aphakic exposure standard limit (H_{Ast-Standard}) as follows:

$$M = {H}_{Ast} / ( {H}_{Ast - Standard} ) \qquad Eq . 6$$

where H_{Ast} and H_{Ast-Standard} are given in mJ/cm².

For example, at each illumination event (or transition) such as t₂, t₃, t₄, t₅, etc., the peak total aphakic exposure (H_{Ast-i}) may be determined and presented to the user via a GUI displayed on touchscreen display 114, such as H_{Ast-1} 512, H_{Ast-2} 522, H_{Ast-3} 532, H_{Ast-4} 542, etc. The requirement for the relevant aphakic exposure standard limit (H_{Ast-Standard}) 600 may also be displayed as well, such as ISO Standard 15752:2010 (10,000 mJ/cm² in 30 minutes). Additionally, in response to the user's request during the surgical procedure, such as at time t_{User}, the peak total aphakic exposure may be determined and presented to the user via a GUI displayed on touchscreen display 114, such as H_{Ast-User} 552. The aphakic figure of merit (Mᵢ) 560 for the most-recently calculated peak total aphakic exposure (H_{Ast-i}) may be displayed as well. Generally, these data may be displayed in graph and/or numerical format.

In certain embodiments, the peak total aphakic exposure for each illumination event (H_{Ast-i}) may be calculated by integrating the peak total aphakic irradiance (E_{Ast-i}) over the illumination event (Δtᵢ), and then adding the peak total aphakic exposure for the prior illumination event (H_{Ast-i-1}). For the first event, the peak total aphakic exposure for the prior illumination event (H_{Ast-0}) is 0 because no illumination probes were turned on.

For example, the peak total aphakic exposure (H_{Ast-1}) for the first illumination event 410 may be calculated when the second event begins at t₂:

$${H}_{Ast - 1} = {E}_{Ast - 1} \cdot ( Δ {t}_{1} ) + 0 \qquad Eq . 7$$

where E_{Ast-1} is a constant value. Other integration techniques may be used when E_{Ast-1} is not a constant value. The peak total aphakic exposure (H_{Ast-2}) for the second illumination event 420 may be calculated when the third event begins at t₃:

$${H}_{Ast - 2} = {E}_{Ast - 2} \cdot ( Δ {t}_{2} ) + {H}_{Ast - 1} \qquad Eq . 8$$

where E_{Ast-2} is a constant value. Other integration techniques may be used when E_{Ast-2} is a not constant value. Similarly, the peak total aphakic exposure (H_{Ast-3}) for the third illumination event 430 may be calculated when the fourth event begins at t₄:

$${H}_{Ast - 3} = {E}_{Ast - 3} \cdot ( Δ {t}_{3} ) + {H}_{Ast - 2} \qquad Eq . 9$$

where E_{Ast-3} is a constant value. Other integration techniques may be used when E_{Ast-3} is a not constant value. Similarly, the peak total aphakic exposure (H_{Ast-4}) for the fourth illumination event 440 may be calculated when the fourth event ends at t₅:

$${H}_{Ast - 4} = {E}_{Ast - 4} \cdot ( Δ {t}_{4} ) + {H}_{Ast - 3} \qquad Eq . 10$$

where E_{Ast-4} is a constant value. Other integration techniques may be used when E_{Ast-4} is a not constant value.

Additionally, in response to the user's request at t_{User}, the peak total aphakic exposure (H_{Ast-User}) may be calculated as follows:

$${H}_{Ast - User} = {E}_{Ast - 4} \cdot ( Δ {t}_{User} ) + {H}_{Ast - 3} \qquad Eq . 11$$

where E_{Ast-4} is a constant value. Other integration techniques may be used when E_{Ast-4} is a not constant value.

In certain embodiments, the aphakic hazard metric may include a projected remaining aphakic exposure time (t_{Standard}). In response to a user request, the projected remaining aphakic exposure time (t_{Standard}) before the relevant aphakic exposure standard limit (H_{Ast-Standard}) is reached may be determined and presented to the user via a GUI displayed on touchscreen display 114, such as 10.5 minutes, 7 minutes, etc. In other embodiments, the projected remaining aphakic exposure time (t_{Standard-i}) may be determined and presented to the user via the GUI at the beginning of each illumination event.

For example, the projected remaining aphakic exposure time (t_{Standard-i}) before the current peak total aphakic exposure (H_{Ast-i}) reaches the relevant aphakic exposure standard limit (H_{Ast-Standard}) may be calculated by subtracting the aphakic exposure for the prior illumination event (H_{Ast-i-1}) from the relevant aphakic exposure standard limit (H_{Ast-Standard}), and then dividing this number by the current peak total aphakic irradiance (E_{Ast-i}). A negative result means that the relevant aphakic exposure standard limit (H_{Ast-Standard}) has been met or exceeded.

FIG. 5B depicts a graph 501 of the peak total aphakic exposure (H_{Ast}) 550 over time (as depicted in FIG. 5A). Time (minutes) is presented on the X-axis, while peak total aphakic exposure (H_{Ast}, mJ/cm²) is presented on the Y-axis.

During the first illumination event 410, the peak total aphakic exposure for the prior illumination event (H_{Ast-0}) is 0 because no illumination probes were turned on. Accordingly, the projected remaining aphakic exposure time (t_{Standard-1}) 610 before the current peak total aphakic irradiance (E_{Ast-1}) 412 reaches the relevant aphakic exposure standard limit (H_{Ast-Standard}) may be calculated as follows:

$${t}_{Standard - 1} = ( {H}_{Ast - Standard} - 0 ) / {E}_{Ast - 1} \qquad Eq . 12$$

The projected remaining aphakic exposure time (t_{Standard-1}) 610 is greater than t₅, which is the end of the illumination for the surgical procedure in this example. In other words, the first illumination event 410 may continue at peak total aphakic irradiance (E_{Ast-1}) 412 throughout the surgical procedure without the risk of excessive aphakic exposure for the patient.

FIG. 5C depicts a graph 502 of the peak total aphakic exposure (H_{Ast}) 550 over time (as depicted in FIGS. 5A, 5B). Time (minutes) is presented on the X-axis, while peak total aphakic exposure (H_{Ast}, mJ/cm²) is presented on the Y-axis.

During the second illumination event 420, the projected remaining aphakic exposure time (t_{Standard-2}) 620 before the current peak total aphakic irradiance (E_{Ast-2}) 422 reaches the relevant aphakic exposure standard limit (H_{Ast-Standard}) may be calculated as follows:

$${t}_{Standard - 2} = ( {H}_{Ast - Standard} - {H}_{Ast - 1} ) / {E}_{Ast - 2} \qquad Eq . 13$$

The projected remaining aphakic exposure time (t_{Standard-2}) 620 is significantly less than t₅, which is the end of the illumination for the surgical procedure in this example. In other words, the second illumination event 420 may only continue at peak total aphakic irradiance (E_{Ast-2}) 422 for a short period of time until excessive aphakic exposure becomes a risk for the patient.

FIG. 5D depicts a graph 503 of the peak total aphakic exposure (H_{Ast}) 550 over time (as depicted in FIGS. 5A, 5B, 5C). Time (minutes) is presented on the X-axis, while peak total aphakic exposure (H_{Ast}, mJ/cm²) is presented on the Y-axis.

During the third illumination event 430, the projected remaining aphakic exposure time (t_{Standard-3}) 630 before the current peak total aphakic irradiance (E_{Ast-3}) 432 reaches the relevant aphakic exposure standard limit (H_{Ast-Standard}) may be calculated as follows:

$${t}_{Standard - 3} = ( {H}_{Ast - Standard} - {H}_{Ast - 2} ) / {E}_{Ast - 3} \qquad Eq . 13$$

The projected remaining aphakic exposure time (t_{Standard-3}) 630 is, again, significantly less than t₅, which is the end of the illumination for the surgical procedure in this example. In other words, the third illumination event 430 may only continue at peak total aphakic irradiance (E_{Ast-3}) 432 for a relatively short period of time until excessive aphakic exposure becomes a risk for the patient.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A method for controlling illumination light sources, the method comprising:
receiving a request to change an illumination output for one or more illumination light sources, the request including a requested light level (L_{requested});
determining a total aphakic irradiance (Eₐₛₜ) for the one or more illumination light sources;
determining a total aphakic irradiance normalized standard deviation (σ_{Eat}′) for the one or more illumination light sources;
determining a peak total aphakic irradiance (E_{Ast}) based on the total aphakic irradiance (Eₐₛₜ) and the total aphakic irradiance normalized standard deviation (σ_{Eat}′);
when the peak total aphakic irradiance (E_{Ast}) is less than or equal to an aphakic irradiance limit (E_{Limit}), changing the illumination output of the one or more illumination light sources based on the requested light level (L_{requested}); and
when the peak total aphakic irradiance (E_{Ast}) is greater than the aphakic irradiance limit (E_{Limit}):
reducing the requested light level (L_{requested}) until the peak total aphakic irradiance (E_{Ast}) is less than or equal to the aphakic irradiance limit (E_{Limit}), and
changing the illumination output of the one or more illumination light sources based on the reduced requested light level (L_{reduced}).

2. The method of claim 1, wherein determining the total aphakic irradiance (Eₐₛₜ) comprises:
determining an aphakic irradiance (Eₐₛ) for each illumination light source; and
summing the aphakic irradiance (Eₐₛ) for each illumination light source to generate the total aphakic irradiance (Eₐₛₜ).

3. The method of claim 2, wherein the aphakic irradiance (Eₐₛ) for each illumination light source is determined by:
determining a light level ratio by dividing the requested light level (L_{requested}) by a maximum light level (Lₘₐₓ); and
multiplying the light level ratio by a maximum light level aphakic irradiance (Eₐₛ₋ₘₐₓ) to generate the aphakic irradiance (Eₐₛ).

4. The method of claim 2, wherein determining the total aphakic irradiance normalized standard deviation (σ_{Eat}′) comprises:
determining an aphakic irradiance normalized standard deviation (σ_{Ea}′) for each illumination light source; and
determining a square root of a sum of the squares of the aphakic irradiance normalized standard deviation (σ_{Ea}′) for each illumination light source to generate the total aphakic irradiance normalized standard deviation (σ_{Eat}′).

5. The method of claim 4, wherein determining the aphakic irradiance normalized standard deviation (σ_{Ea}′) for each illumination light source comprises:
determining an aphakic irradiance standard deviation (σ_{Ea}); and
dividing the aphakic irradiance standard deviation (σ_{Ea}) by the aphakic irradiance (Eₐₛ) to generate the aphakic irradiance normalized standard deviation (σ_{Ea}′).

6. The method of claim 5, wherein determining the peak total aphakic irradiance (E_{Ast}) comprises:
multiplying the aphakic irradiance normalized standard deviation (σ_{Ea}′) by a safety factor (n) to generate a first value (n·σ_{Eat}′);
adding 1 to the first value to generate a second value (1+n·σ_{Eat}′); and
multiplying the second value by the total aphakic irradiance (Eₐₛₜ) to generate the peak total aphakic irradiance (E_{Ast}=Eₐₛₜ·(1+n·σ_{Eat}′).

7. The method of claim 6, wherein the safety factor (n) equals 4.75.

8. The method of claim 1, wherein:
reducing the requested light level (L_{requested}) until the peak total aphakic irradiance (E_{Ast}) is less than or equal to the aphakic irradiance limit (E_{Limit}) comprises:
dividing the peak total aphakic irradiance (E_{Ast}) by the aphakic irradiance limit (E_{Limit}) to generate an aphakic irradiance ratio (E_{Ast}/E_{Limit}), and
dividing the requested light level (L_{requested}) by the aphakic irradiance ratio (E_{Ast}/E_{Limit}) to generate the reduced requested light level (L_{reduced}).

9. The method of claim 1, wherein:
each of the one or more illumination light sources includes a red light emitting diode (LED), a green LED, and a blue LED; and
reducing the requested light level (L_{requested}) until the peak total aphakic irradiance (E_{Ast}) is less than or equal to the aphakic irradiance limit (E_{Limit}) comprises:
reducing a blue LED light level (L_{B}) of each illumination light source to zero,
reducing a green LED light level (L_{G}) of each illumination light source until the peak total aphakic irradiance (E_{Ast}) is less than or equal to the aphakic irradiance limit (E_{Limit}) to generate the reduced requested light level (L_{reduced}).

10. The method of claim 1, wherein:
each of the one or more illumination light sources includes a white light emitting diode (LED); and
reducing the requested light level (L_{requested}) until the peak total aphakic irradiance (E_{Ast}) is less than or equal to the aphakic irradiance limit (E_{Limit}) comprises:
inserting a blue-blocking longpass filter into a light path of each illumination light source, and
reducing a white LED light level (L_{W}) of each illumination light source until the peak total aphakic irradiance (E_{Ast}) is less than or equal to the aphakic irradiance limit (E_{Limit}) to generate the reduced requested light level (L_{reduced}).

11. A surgical system, comprising:
one or more illumination light sources;
a processor, coupled to the illumination light sources, the processor configured to:
receive a request to change an illumination output for the one or more illumination light sources, the request including a requested light level (L_{requested});
determine a total aphakic irradiance (Eₐₛₜ) for the one or more illumination light sources;
determine a total aphakic irradiance normalized standard deviation (σ_{Eat}′) for the one or more illumination light sources;
determine a peak total aphakic irradiance (E_{Ast}) based on the total aphakic irradiance (Eₐₛₜ) and the total aphakic irradiance normalized standard deviation (σ_{Eat}′);
when the peak total aphakic irradiance (E_{Ast}) is less than or equal to an aphakic irradiance limit (E_{Limit}), change the illumination output of the one or more illumination light sources based on the requested light level (L_{requested}); and
when the peak total aphakic irradiance (E_{Ast}) is greater than the aphakic irradiance limit (E_{Limit}):
reduce the requested light level (L_{requested}) until the peak total aphakic irradiance (E_{Ast}) is less than or equal to the aphakic irradiance limit (E_{Limit}), and
change the illumination output of the one or more illumination light sources based on the reduced requested light level (L_{reduced}).

12. The system of claim 11, wherein:
said reduce the requested light level (L_{requested}) comprises:
divide the peak total aphakic irradiance (E_{Ast}) by the aphakic irradiance limit (E_{Limit}) to generate an aphakic irradiance ratio (E_{Ast}/E_{Limit}); and
divide the requested light level (L_{requested}) by the aphakic irradiance ratio (E_{Ast}/E_{Limit}) to generate the reduced requested light level (L_{reduced}).

13. The system of claim 11, wherein:
each of the one or more illumination light sources includes a red light emitting diode (LED), a green LED, and a blue LED; and
said reduce the requested light level (L_{requested}) comprises:
reduce a blue LED light level (L_{B}) of each illumination light source to zero,
reduce a green LED light level (L_{G}) of each illumination light source until the peak total aphakic irradiance (E_{Ast}) is less than or equal to the aphakic irradiance limit (E_{Limit}) to generate the reduced requested light level (L_{reduced}).

14. The system of claim 11, wherein:
each of the one or more illumination light sources includes a white light emitting diode (LED); and
said reduce the requested light level (L_{requested}) comprises:
insert a blue-blocking longpass filter into a light path of each illumination light source, and
reduce a white LED light level (L_{W}) of each illumination light source until the peak total aphakic irradiance (E_{Ast}) is less than or equal to the aphakic irradiance limit (E_{Limit}) to generate the reduced requested light level (L_{reduced}).

15. The system of claim 11, wherein the processor is further configured to:
for each illumination light source:
determine an aphakic irradiance (Eₐₛ),
determine an aphakic irradiance standard deviation (σ_{Ea}), and
determine an aphakic irradiance normalized standard deviation (σ_{Ea}′) by dividing the aphakic irradiance standard deviation (σ_{Ea}) by the aphakic irradiance (Eₐₛ);
generate the total aphakic irradiance (Eₐₛₜ) by summing the aphakic irradiance (Eₐₛ) for each illumination light source;
generate the total aphakic irradiance normalized standard deviation (σ_{Eat}′) by determining a square root of a sum of the squares of the aphakic irradiance normalized standard deviation (σ_{Ea}′) for each illumination light source; and
generate the peak total aphakic irradiance (E_{Ast}=Eₐₛₜ·(1+n·σ_{Eat}′)) by:
multiplying the aphakic irradiance normalized standard deviation (σ_{Ea}′) by a safety factor (n) to generating a first value (n·σ_{Eat}′), where the safety factor is equal to 4.75,
adding 1 to the first value to generate a second value (1+n·Eat′), and
multiplying the second value by the total aphakic irradiance (Eₐₛₜ).
